(19) United States
(12) Patent Application Publication
(10) Pub. No.: US 2017/0290510 A1
(43) Pub. Date: Oct. 12, 2017

(54) FETAL PULSE OXIMETERS AND METHODS OF USING THE SAME

(71) Applicant: Baylor College of Medicine, Houston, TX (US)

(72) Inventors: Magdalena Sanz Cortes, Houston, TX (US); Claudia Iriondo, Houston, TX (US); Kathryn Wallace, Lithia, FL (US); Thomas Loughlin, Houston, TX (US); Samir Saidi, Dallas, TX (US)

(21) Appl. No.: 15/477,648

(22) Filed: Apr. 3, 2017

Related U.S. Application Data
(60) Provisional application No. 62/321,322, filed on Apr. 12, 2016.

(51) Int. Cl. A61B 5/00; A61B 5/145; A61B 5/1455; A61B 5/1464

(52) U.S. Cl. CPC A61B 5/00; A61B 5/1464; A61B 5/145; A61B 5/1455; A61B 5/14551; A61B 5/14552; A61B 2503/02; A61B 5/4362

(57) ABSTRACT

One aspect of the invention provides a fetal pulse oximeter including: a shape-memory member adapted and configured to expand outward and define a loop when advanced out of a cannula; one or more light sources mounted on the shape memory member and facing toward a center of the loop, the one or more light sources adapted and configured to generate red and infrared light; and a photodiode mounted on the shape memory member and facing toward a center of the loop. Another aspect of the invention provides a method for measuring recording pulse and blood oxygen saturation. The method includes: advancing the fetal pulse oximeter as described herein out of a cannula within a placenta; allowing the shape-memory member to expand outward; and placing the loop over a limb.

### CROSS-REFERENCE TO RELATED APPLICATION

This application claims the benefit of priority under 35 U.S.C. §119(e) of U.S. Provisional Patent Application Ser. No. 62/321,322, filed Apr. 12, 2016. The entire content of this application is hereby incorporated by reference herein.

### BACKGROUND OF THE INVENTION

Fetuses during fetoscopic surgery can suffer from hypoxia, which can cause permanent organ damage or death. If detected early, hypoxia can easily be addressed by physical or chemical means. However, there is no viable method to monitor oxygen levels of fetuses during fetoscopic surgery. Currently, pulse oximeters are too large (by at least about 50×) to pass through the 3 mm diameter trocar used to gain access to the fetus during the surgery.

### SUMMARY OF THE INVENTION

One aspect of the invention provides a fetal pulse oximeter including: a shape-memory member adapted and configured to expand outward and define a loop when advanced out of a cannula; one or more light sources mounted on the shape memory member and facing toward a center of the loop, the one or more light sources adapted and configured to generate red and infrared light; and a photodiode mounted on the shape memory member and facing toward a center of the loop.

This aspect of the invention can have a variety of embodiments. The shape-memory member can be adapted and configured to expand to have a sinusoidal region.

The shape-memory member can exhibit temperature-dependent expansion. The shape-memory member can exhibit temperature-dependent expansion at a temperature between about 20° C. and about 40° C. The shape-memory member can exhibit temperature-dependent expansion at a temperature between about 30° C. and about 37° C. The shape-memory member can exhibit temperature-dependent expansion at a temperature between about 35° C. and about 37° C. The shape-memory member can exhibit temperature-dependent expansion at a temperature of about 37° C.

The shape-memory member can be nitinol.

The loop can have a cross-sectional dimension D of between about 1 cm and about 3 cm.

Another aspect of the invention provides a method for measuring or recording pulse and blood oxygen saturation. The method includes: advancing a fetal pulse oximeter as described herein out of a cannula within a placenta; allowing the shape-memory member to expand outward; and placing the loop over a limb.

This aspect of the invention can have a variety of embodiments. The method can further include tightening the loop to press the one or more light sources and the photodiode against the surface of the limb.

The method can further include coupling lead wires from the one or more light sources and the photodiode to a vital signs monitor. The method can further include actuating the vital signs monitor to: selectively actuate the one or more light sources; receive one or more signals from the photodiode; and calculate oxygen saturation (SO₂) as a function of at least the one or more signals received from the photodiode after emission by the one or more light sources. The method can further include loosening the loop. The method can further include cutting the loop. The method can further include removing the loop from the placenta.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1 depicts a fetal pulse oximeter according to an embodiment of the invention.

FIG. 2 depicts a method of measuring pulse and blood oxygen concentration according to an embodiment of the invention.

FIG. 3 depicts a method for measuring pulse and blood oxygen saturation according to an embodiment of the invention.

### DEFINITIONS

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION OF THE INVENTION

Embodiments of the invention provide fetal pulse oximeters and methods of use thereof.

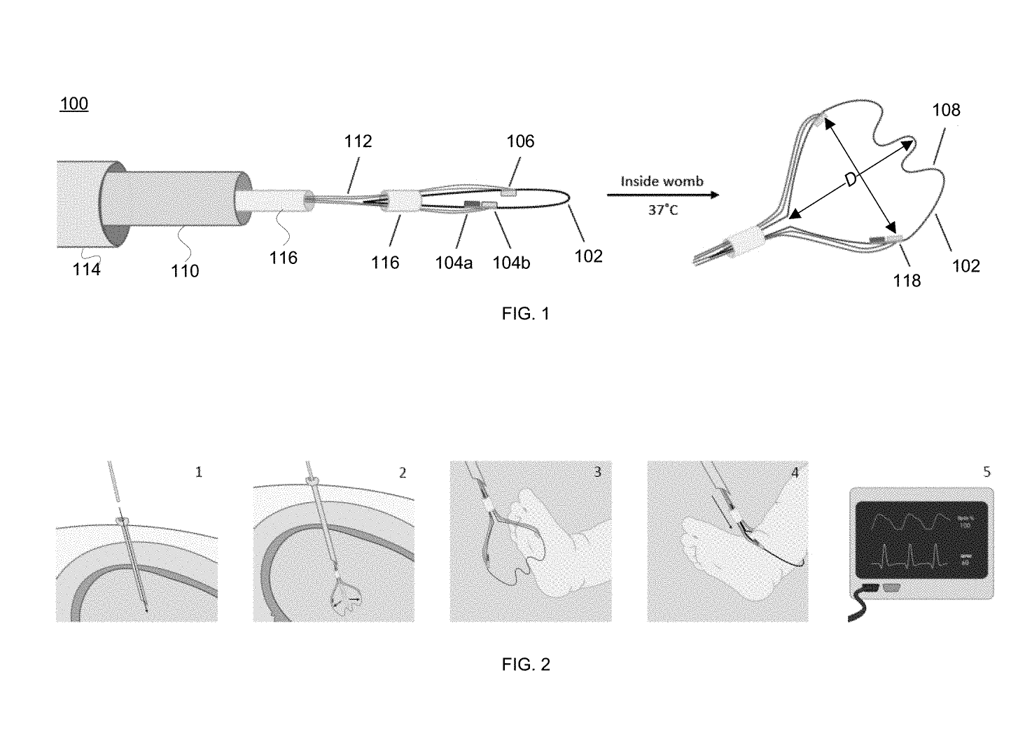

Referring now to FIG. 1, one embodiment of the invention provides a fetal pulse oximeter 100. The fetal pulse oximeter 100 can include a shape-memory member 102, one or more light sources 104a, 104b, and a photodetector 106.

The shape-memory member 102 can be adapted and configured to expand to define an internal opening sufficient to encircle a fetal limb (e.g., an arm or a leg). The shape-memory member 102 can expand to a variety of profiles (e.g., a substantially circular profile). For example, the shape-memory member 102 can be adapted and configured to expand to accommodate a cross-sectional dimension D of between about 1 cm and about 3 cm.

Shape-memory member 102 can be fabricated from a variety of materials such as shape-memory alloys such as nickel-titanium alloys (colloquially known as “nitinol”). Other exemplary shape-memory alloys include Ag—Cd, Au—Cd, Cu—Al—Ni, Cu—Sn, Cu—Zn, Cu—Zn—X (X=Si, Al, Sn), Fe—Pt, Mn—Cu, Fe—Mn—Si, Co—Ni—Al, Co—Ni—Ga, Ni—Fe—Ga, Ti—Nb, Ni—Ti—Hf, Ni—Ti—Pd, and Ni—Mn—Ga.

The shape-memory member 102 can have a one-way memory effect and expand to the desired dimensions at a variety of temperatures (e.g., room temperature) or have a two-way memory effect and exhibit temperature-dependent expansion (e.g., at a temperature typically found in the womb such as about 37° C.). In one embodiment, the shape-memory member 102 is adapted and configured to change from a folded arrangement shown on the left of FIG. 1 to looped arrangement as shown in the right of FIG. 1. Such a transformation can be facilitated by the configuration of a central region 108 to take on a sinusoidal shape upon deployment within the womb. Shape-memory member 102 can be bound or coupled to form a closed loop as depicted in FIG. 1 or can be open.

The sinusoidal-shaped central region 108 can provide visual feedback (e.g., through imaging modalities such as ultrasound) to a user to prevent overtightening of the collar, shape-memory member 102, which would cause the sinusoidal-shaped central region 108 to straighten.

In one embodiment, a 20 cm length of body temperature nitinol is shaped in a sinusoidal pattern along an arc of diameter 5 cm. Body temperature nitinol has an Active A(f) temperature—specifying the completion of the shape recovery transformation upon heating—between about 20° C. and about 40° C. (±5° C.) and is available from Johnson Matthey of West Chester, Pa.

The shape-memory member 102 can serve as a scaffold along which other components such as light sources 104a, 104b and photodetector 106 can be mounted. Mounting can occur through mechanical fasteners (e.g., staples), chemical fasteners (e.g., adhesives), soldering, and the like.

Light sources 104a, 104b can be adapted and configured to produce both red and infrared wavelengths of light. For example, the light sources 104a, 104b can produce red light having a wavelength of between about 600 nm and about 750 nm (e.g., about 650 mm, about 660 nm, and the like) and infrared light having a wavelength of about 850 mm and about 1,000 nm (e.g., about 900 nm, about 910 nm, about 940 nm, about 950 mm, and the like). Other suitable LED wavelengths are described in John TB Moyle, Pulse Oximetry 16-21 (2d ed. 2002).

Light sources 104a, 104b can be separate light sources. Alternatively, a single light source can provide light at both red and infrared wavelengths (e.g., through the use of filters). If separate, light sources 104a, 104b are preferably proximate or adjacent to each other so that the optical path from light sources 104a, 104b to photodetector 106 is substantially similar (e.g., in length and/or angle).

Light sources 104a, 104b can be any device capable of producing the desired wavelengths, fitting within a cannula 110, and operating within a womb. Suitable light sources include light-emitting diodes (LEDs) such as 0805 LEDs available from sources such as OSRAM Opto Semiconductors Inc. of Sunnyvale, Calif. Such 0805 LEDs have a length of about 2 mm, a width of about 1.25 mm, and a height of about 0.8 mm.

Photodetector 106 can be arranged on an opposite (e.g., diametrically opposite) side of the shape-memory member 102 in order to measure red and infrared light passing through the fetal limb held within the shape-memory member 102. Various sizes of fetal pulse oximeter 100 can be produced in order to accommodate limbs of various sizes so that light sources 104a, 104b and photodetector 106 will be positioned on opposite sides of various size limbs. In another embodiment, a fetal pulse oximeter 100 can include an array of photodetectors 106 so that a well-positioned photodetector 106 (e.g., a photodetector 106 located on an opposite side of the limb from light sources 104a, 104b) can be selected and monitored after placement over the limb.

Although photodetector 106 is depicted on an opposite side of the shape-memory member 102 from light sources 104a, 104b, embodiments of the invention can also implement reflectance and/or transflectance pulse oximetry in which the light sources 104a, 104b and the photodetector 106 are on the same side of the limb, as illustrated in U.S. Pat. Nos. 6,763,256, 8,818,476, and 9,314,197.

Photodetector 106 can be a photodiode such as an 0805 photodiode such as the TEMD7000X01 available from Vishay Semiconductors of Malvern, Pa.

Light sources 104a, 104b and/or photodetector 106 can be mounted directly to the shape-memory member 102 or can be mounted via a substrate such as a printed circuit board (PCB) such as a flexible PCB 118. Flexible PCB materials are available from sources such as Flexible Circuit Technologies, Inc. of Minneapolis, Minn. In one embodiment, a piece of 3 mm×2.5 mm flexible printed circuit board is used to mount each of light sources 104a, 104b and photodetector 106.

Light sources 104a, 104b and photodetector 106 can be powered and/or communicate via one or more wires 112. For example, a first pair of wires (e.g., 32-gauge wires) can power light sources 104a, 104b and a second pair of wires (e.g., 32-gauge wires) can power and/or transmit measured values from photodetector 106.

As seen in the left of FIG. 1, the fetal pulse oximeter 100 can be packed flat to fit to fit within a cannula 110. For example, cannula 110 can have a 3 mm inner diameter, and the fetal pulse oximeter 100 can have a width of about 2.5 mm and a folded height of about 2.1 mm.

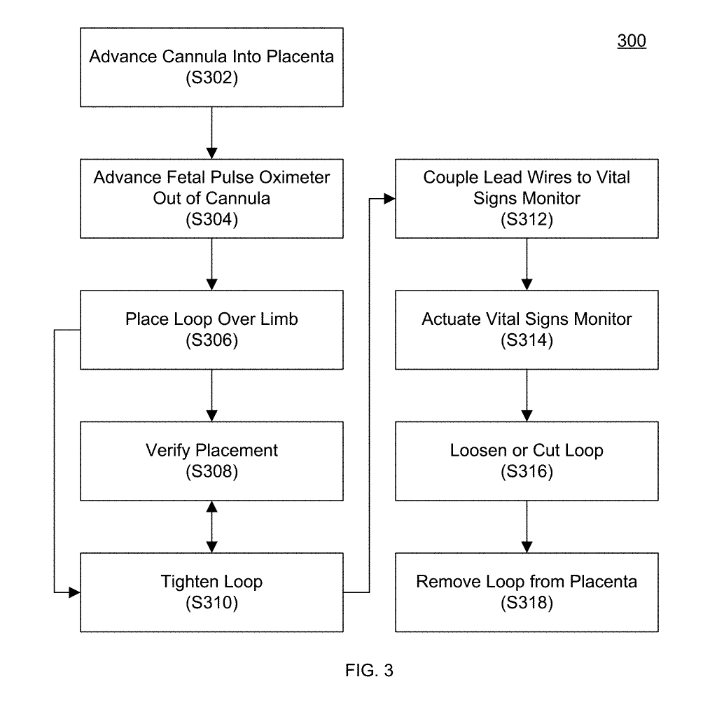

Referring now to FIGS. 2 and 3, cannula 110 can be placed within a trocar 114 for placement within a placenta. After accessing the placenta (S302) in FIG. 2, Panel 1, a pusher cannula 116 can advance the folded fetal pulse oximeter 100 beyond the trocar 114 (S304). In FIG. 2, Panel 2, the shape-memory member 102 expands to define a loop as seen in FIG. 2, Panel 3. In FIG. 2, Panel 4 the loop is placed over a limb (e.g., an ankle) (S306), for example, using various imaging modalities such as endoscopy, fetoscopy, ultrasound, and the like (S308). Pusher cannula 116 can advance a collar 118 to tighten the shape-memory member 102 such that the light sources 104a, 104b and photodetector 106 press against the limb (S310). A compression spring can be included on either side of collar 118 to provide tactile feedback regarding tension applied to the shape-memory member 102. Collar 118 can be sized and/or include a camming or ratcheting device to retain the collar 118 after advancement. In FIG. 2, Panel 5, the wires 112 can be coupled to one or devices for monitoring, displaying, and/or recording pulse and blood oxygen concentration, e.g., using a DB9 connector (S312, S314). Suitable devices include vital sign monitors such as those available from Welch-Allyn of Skaneateles Falls, N.Y. Trocar 114, cannula 110, and/or pusher cannula 116 can be optionally removed by sliding each cylinder back over wires 114 before the wires are coupled to a monitoring device.

Embodiments of the invention can be used as a blood oxygen monitor for fetuses during minimally invasive fetal surgery. A pulse oximeter 100 can loop around the fetal extremity at the beginning of the procedure and remain in place throughout, notifying surgeons if fetal blood oxygenation level falls below the normal range, and allowing them to enact emergency procedures accordingly. Once the procedure is complete, the device 100 can easily be loosened and looped off or cut off (S316) using standard fetoscopic forceps and scissors and withdrawn from the placenta (S318), e.g., by withdrawal through cannula 110.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A fetal pulse oximeter comprising:
a shape-memory member adapted and configured to expand outward and define a loop when advanced out of a cannula;
one or more light sources mounted on the shape memory member and facing toward a center of the loop, the one or more light sources adapted and configured to generate red and infrared light; and
a photodiode mounted on the shape memory member and facing toward a center of the loop.

2. The fetal pulse oximeter of claim 1, wherein the shape-memory member is adapted and configured to expand to have a sinusoidal region.

3. The fetal pulse oximeter of claim 1, wherein the shape-memory member exhibits temperature-dependent expansion.

4. The fetal pulse oximeter of claim 1, wherein the shape-memory member exhibits temperature-dependent expansion at a temperature between about 20° C. and about 40° C.

5. The fetal pulse oximeter of claim 1, wherein the shape-memory member exhibits temperature-dependent expansion at a temperature between about 30° C. and about 37° C.

6. The fetal pulse oximeter of claim 1, wherein the shape-memory member exhibits temperature-dependent expansion at a temperature between about 35° C. and about 37° C.

7. The fetal pulse oximeter of claim 1, wherein the shape-memory member exhibits temperature-dependent expansion at a temperature of about 37° C.

8. The fetal pulse oximeter of claim 1, wherein the shape-memory member is nitinol.

9. The fetal pulse oximeter of claim 1, wherein the loop has a cross-sectional dimension D of between about 1 cm and about 3 cm.

10. A method for measuring or recording pulse and blood oxygen saturation, the method comprising:
advancing the fetal pulse oximeter of claim 1 out of a cannula within a placenta;
allowing the shape-memory member to expand outward; and
placing the loop over a limb.

11. The method of claim 10, further comprising:
tightening the loop to press the one or more light sources and the photodiode against the surface of the limb.

12. The method of claim 10, further comprising:
coupling lead wires from the one or more light sources and the photodiode to a vital signs monitor.

13. The method of claim 12, further comprising:
actuating the vital signs monitor to:
selectively actuate the one or more light sources;
receive one or more signals from the photodiode; and
calculate oxygen saturation (SO₂) as a function of at least the one or more signals received from the photodiode after emission by the one or more light sources.

14. The method of claim 10, further comprising:
loosening the loop.

15. The method of claim 10, further comprising:
cutting the loop.

16. The method of claim 10, further comprising:
removing the loop from the placenta.
